(19) United States
(12) Patent Application Publication
(10) Pub. No.: US 2026/0345005 A1
(43) Pub. Date: Nov. 5, 2026

(54) SYSTEMS, METHODS, AND APPARATUS FOR MONITORING INTRAOCULAR PRESSURE OF AN EYE

(71) Applicant: Johnson & Johnson Surgical Vision, Inc., Irvine, CA (US)

(72) Inventors: Deep Mehta, Irvine, CA (US); Edith Fung, Diamond Bar, CA (US)

(21) Appl. No.: 19/197,832

(22) Filed: May 2, 2025

(51) Int. Cl. A61B 3/16; A61F 9/007

(52) U.S. Cl. CPC A61B 3/16; A61F 9/00745

(57) ABSTRACT

A surgical system for monitoring pressure events of an eye of a patient during a phacoemulsification procedure is disclosed. The system may include at least one sensor configured to produce one or more signals corresponding to pressure of the eye during the phacoemulsification procedures. The system may also include a controller of a surgical console configured to receive the one or more signals from the at least one sensor, determine a deviation between a target value and the pressure associated with each of the one or more received signals, determine a pressure metric based on a function involving the deviations over a period of time, and provide the pressure metric for display on a graphical user interface.

### FIELD

The present disclosure relates generally to the field of ocular surgery and more particularly, to a phacoemulsification system for monitoring and evaluating intraocular pressure (IOP) within the anterior chamber of the eye.

### BACKGROUND

During ophthalmic surgery, an ophthalmic surgical apparatus is used to perform surgical procedures in a patient's eye. An ophthalmic surgical apparatus typically includes a control module, a power supply, an irrigation source, one or more irrigation and/or aspiration pumps, as well as associated electronic hardware and software for operating a multifunction handheld surgical tool or handpiece. The control module of the surgical apparatus may include operating controls for regulating settings or functions of the apparatus and handpiece. The handpiece may include a tip or needle, which may be ultrasonically driven once placed within the incision to, for example, emulsify the lens of the eye, and an irrigation sleeve that coaxially surrounds at least a portion of the needle or tip for supplying irrigation fluid to the eye. In various surgical procedures, the components of the surgical apparatus work together in order to, for example, emulsify the crystalline lens, irrigate the eye with a balanced salt solution, and aspirate the emulsified lens from the eye.

An exemplary type of ophthalmic surgery is phacoemulsification. Phacoemulsification includes making a corneal and/or scleral incision in an eye and inserting a needle or tip of a phacoemulsification handpiece that is ultrasonically driven to emulsify or liquefy the lens. A phacoemulsification system typically includes a handpiece coupled to an irrigation source and an aspiration pump. The handpiece may include a distal tip that emits ultrasonic energy to emulsify a crystalline lens within the patient's eye. The sleeve coupled with the handpiece may include one or more irrigation ports proximal to the distal end and coupled with the irrigation source via an irrigation input line. The needle coupled with the handpiece includes an aspiration port at the distal tip that is coupled with the aspiration pump via an aspiration output line. Concomitantly with the emulsification, fluid from the irrigation source (e.g., a bottle or bag of balanced salt solution that may be elevated above the patient's eye to establish positive pressure by gravity, and/or with an external pressure source) is irrigated into the eye via the irrigation line and the irrigation port(s). The fluid is directed to the crystalline lens in the patient's eye in order to maintain pressure in the anterior chamber and to replenish the fluid aspirated from the eye. The irrigation fluid in the patient's eye and the crystalline lens material may be aspirated or removed from the eye by the aspiration pump and line via the aspiration port. In some instances, the aspiration pump may be in the form of, for example, a peristaltic or positive displacement pump. Other forms of aspiration pumps are well known in the art, such as vacuum pumps (e.g., a Venturi pump). In addition, more than one pump and/or more than one type of pump may be used. Additionally, some procedures may include irrigating the eye and aspirating the irrigation fluid without concomitant destruction, alteration or removal of the lens.

Intraocular Pressure (IOP) is the fluid pressure inside the anterior chamber of the eye. In a normal eye, IOP may vary depending on the time of day, activities of the patient, fluid intake, medications, etc. IOP may be measured as static (e.g., a specific value) or dynamic (e.g., a range of values). As can be appreciated, the IOP (e.g., the static IOP and/or the dynamic IOP) of a patient's eye can fluctuate greatly during an ophthalmic surgery procedure. It is well-known that the IOP in an anterior chamber of the eye is required to be controlled and maintained during a surgical procedure in order to avoid damage to the structures of the eye. For the correct function of the eye and its structure (e.g., shape) and to preserve sharp and undamaged vision, it is important to maintain the intraoperative IOP at or near a desired or target value.

Different medically recognized techniques have been utilized for ophthalmic surgery, such as phacoemulsification, in order to maintain and control the intraoperative IOP of a patient's eye. In various examples, phacoemulsification may involve combining irrigation, aspiration, and emulsification within a single handpiece. The handpiece may be typically controlled electrically in order to, for example, control the flow of fluid through the handpiece and the tip. As may be appreciated, during a surgical procedure, the flow of fluid to and from a patient's eye (through a fluid infusion/irrigation system or aspiration/extraction system, for example), the fluid pressure flowing through the handpiece, and the power control over the handpiece, are all critical to the procedure performed.

The aspiration and irrigation to the crystalline lens may be controlled in order maintain a desired or optimal IOP within the anterior chamber of the eye and the operation of the ultrasonic energy of the handpiece may be used as little as possible to avoid causing damage to the surgical site during the procedure. Similarly, it may be necessary to maintain a stable volume of liquid in the anterior chamber of the eye, which may be accomplished by irrigating fluid into the eye at the same rate as aspirating fluid and lens material from the eye. Accordingly, the ability to assess the IOP of a patient's eye during a surgical operation would be beneficial to a surgeon or user when operating a surgical system for phacoemulsification surgery.

In traditional phacoemulsification surgical systems, the parameters and/or settings of the system may be electronically controlled or modified by a surgeon and/or by a computer system or control module. For instance, a control module may provide feedback information to a surgeon or user regarding the function and operation of the system and/or may receive input from a user or surgeon in order to adjust surgical settings. However, current phacoemulsification systems, including those using a gravity-based infusion system, may not provide any indication(s) relative to surge events, such as IOP surge events. Further, current phacoemulsification systems that control and manage the IOP during the surgical procedure may not be configured to analyze deviations of the IOP from a setpoint or target IOP during the surgical procedure.

Based on the foregoing, it would be beneficial to provide a surgical system for monitoring deviations and fluctuations of the IOP of a patient's eye throughout a surgical operation. Further, it would be advantageous to provide metrics indicating deviations in the IOP over the course of the surgical procedure. Such information may afford a surgeon the ability to perform desired phacoemulsification, diathermy, and/or vitrectomy functions with better understanding of the surgical environment and process during the surgical procedure.

### BRIEF DESCRIPTION OF THE DRAWINGS

The following detailed description should be read with reference to the drawings, in which like elements in different drawings may be identically numbered. The drawings, which are not necessarily to scale, depict selected examples and are not intended to limit the scope of the disclosure. The detailed description illustrates by way of example, not by way of limitation, the principles of the disclosed examples. This description will clearly enable one skilled in the art to make and use the described several examples, adaptations, variations, alternatives, and uses of the disclosure. The detailed description refers to the accompanying figures in which:

FIG. 1 is a schematic, pictorial illustration of an example of a phacoemulsification system;

FIG. 2 illustrates pressure limits and setpoints for a phacoemulsification system, in accordance with one example;

FIG. 3A is a histogram illustrating IOP measurements taken over a range of values;

FIG. 3B is graph illustrating a variability of IOP measurements from a set point;

FIG. 4A is a histogram illustrating IOP measurements taken over a range of values;

FIG. 4B is graph illustrating a variability of IOP measurements from a set point;

FIG. 5A is a histogram illustrating IOP measurements taken over a range of values;

FIG. 5B is graph illustrating a variability of the IOP measurements from a set point;

FIGS. 6A-6C illustrate the results of an experiment of measuring IOP values over time;

FIGS. 7A-7C illustrate the results of an experiment of measuring IOP values over time;

FIG. 8 illustrates a graphical user interface of a surgical system, in accordance with one example;

FIG. 9 illustrates another graphical user interface of a surgical system, in accordance with one example; and

FIG. 10 is a flow chart of a phacoemulsification method, in accordance with one example.

### DETAILED DESCRIPTION

The following description and the drawings provided herein may have been simplified to illustrate aspects that are relevant for a clear understanding of the herein described systems, devices, and methods, while eliminating, for the purpose of clarity, other aspects that may be found in typical similar systems, devices, and methods. Those of ordinary skill may thus recognize that other elements and/or operations may be desirable and/or necessary to implement the systems, devices, and methods described herein. But because such elements and operations are known in the art, and because they may not facilitate a better understanding of the present disclosure, for the sake of brevity a discussion of such elements and operations may not be provided herein. However, the present disclosure is deemed to nevertheless include all such elements, variations, and modifications to the described aspects that would be known to those of ordinary skill in the art.

Examples are provided herein so that this disclosure is sufficiently thorough and fully conveys the scope of the disclosed examples to those who are skilled in the art. Numerous specific details are set forth, such as examples of specific components, devices, and methods, to provide a thorough understanding of examples of the present disclosure. Nevertheless, it will be apparent to those skilled in the art that certain specific disclosed details need not be employed, and that exemplary examples may be embodied in different forms. As such, the exemplary examples should not be construed to limit the scope of the disclosure. As referenced above, in some exemplary examples, well-known processes, well-known device structures, and well-known technologies may not be described in detail.

The present disclosure is directed to surgical systems and methods for monitoring and evaluating the IOP of an anterior chamber of an eye of a patient during phacoemulsification surgery. In particular, the surgical systems may determine deviations and/or fluctuations of the IOP within the eye and provide IOP information to surgeons or users of the surgical systems. The surgical systems may be configured to receive information provided or inputted by a user or surgeon and/or to detect various parameters, variables, and conditions of the surgical system. For example, the surgical systems may include a graphical user interface or other user interface that enables a user to input IOP information (e.g., a target or set point IOP) for a surgical procedure to be performed on a patient's eye. Further, the surgical systems may include one or more sensors to detect information about various parameters, conditions, and/or components of the surgical system, such as pressure information, the rate of fluid flow before and after the fluid flows through a phacoemulsification handpiece, etc.

In one aspect, a surgical system for monitoring pressure events of an eye of a patient during a phacoemulsification procedure is disclosed. The system may include at least one sensor configured to produce one or more signals corresponding to pressure of the eye during the phacoemulsification procedure. The system may also include a controller of a surgical console configured to receive the one or more signals from the at least one sensor, determine a deviation between a target value and the pressure associated with each of the one or more received signals, determine a pressure metric based on a function involving the deviations over a period of time, and provide the pressure metric for display on a graphical user interface.

In another aspect, an ophthalmic surgical system is disclosed. The system may include a phacoemulsification probe for performing a phacoemulsification procedure. The phacoemulsification probe may include a needle and a sleeve at its distal end, the needle configured to be inserted into an eye of a patient. The system may also include a pressure sensor configured to detect a pressure in an eye of a patient. Further, the system may include a processor configured to receive a first sequence of detected signals from the pressure sensor. Each of the received signals may represent a pressure within the eye during the phacoemulsification procedure. The processor may also be configured to determine a clinical index based at least on a function of the received signals. The function may involve a deviation of the pressure associated with each of the received signals from a threshold pressure. Further, the processor may be configured to provide to a user of the surgical system an indication of the clinical index to the patient. In a further aspect, a method for monitoring pressure events during a medical procedure performed on an eye of a patient is disclosed. The method may include receiving a sequence of signals detected by at least one sensor. Each of the received signals may represent pressure of the eye during the medical procedure. The method may also include determining a deviation between the pressure associated with each of the one or more received signals and a target value, determining a pressure metric based on a function involving the deviations, and providing an indication of the clinical index to a user.

In a phacoemulsification procedure, a surgeon uses a phacoemulsification handpiece to break the lens into particles, and the particles may be aspirated from the eye via an aspiration line from the needle coupled with the handpiece. In order to maintain the IOP within the eye at or near a target or setpoint IOP and within acceptable limits or bounds while aspiration occurs, the eye may be separately irrigated via an irrigation line to a sleeve coupled with the handpiece. The irrigation flow and the aspiration flow may be balanced to maintain the IOP of the eye at or near the set point IOP and to prevent the irrigation and aspiration from exceeding acceptable limits or bounds, since breaching either an upper or a lower IOP bound may cause irreparable damage to the eye. For example, if the aspiration is increased to remove cataract particles from the eye, irrigation may need to be increased to compensate for the increase aspiration.

To facilitate nominal and safe phacoemulsification operation, a desired working range of the IOP during phacoemulsification is typically maintained between 20 mmHg and 120 mmHg. Below 20 mmHg, the eye chamber may collapse. Above 120 mmHg, the eye may develop clouding. To allow sufficient chamber depth for a surgeon to perform a given procedure, in some examples, the IOP at the surgical site may be maintained at or around an elevated value of 70 mmHg (while normal physiological IOP is between 11 and 21 mmHg). The IOP may be maintained by controlling the irrigation, the aspiration, or both as further described below.

Typically, during the procedure, the surgical site is irrigated continuously, and the aspiration is toggled as required by the surgeon, typically by the surgeon activating a foot pedal (e.g., from foot pedal position (FP) 1 to FP 2). When the surgeon activates the aspiration with the foot pedal, the activation may typically be followed by an abrupt dip in the IOP. Similarly, a dip in the IOP may also occur during a vacuum surge that may occur post occlusion due to vacuum buildup that occurred during the occlusion and/or based on reactivating the aspiration flow after temporarily cutting off the aspiration to avoid the vacuum surge.

The IOP of a patient's eye may be monitored with one or more sensors (e.g., a pressure sensor) coupled with the irrigation line or otherwise positioned near the surgical site. A controller, typically including a feedback control loop, may be configured to regulate the IOP within the eye and to reduce deviations and/or fluctuations of the IOP. The controller may determine the IOP within the anterior chamber of a patient's eye in real time during a surgical procedure. For example, the controller may receive output from the one or more sensors and compare the output to a setpoint IOP input by a user or stored in memory accessible to the controller. The setpoint IOP is typically a target IOP that is desired to be maintained during the surgical procedure. When a difference occurs between the IOP within the patient's eye and the set point IOP, the controller may dynamically adjust the operation (e.g., parameters) of the surgical system based on the IOP that is currently detected at the surgical site. For example, the controller may be configured to change the flow rate of a pump (e.g., an irrigation or aspiration pump) to compensate for the difference detected and return the IOP to the desired level or set point IOP.

The controller may monitor and evaluate the deviations, variations, and/or fluctuations of the IOP within the patient's eye. For example, the controller may collect and use parameters and conditions relating to the operation of the surgical system to maintain the IOP within the patient's eye at the set point or target IOP. The controller may be configured to maintain the IOP by limiting or reducing the fluctuations of the IOP in the eye.

The surgical system may provide metrics or information relating to the IOP based on an evaluation of the IOP within the patient's eye over a time period. For example, the surgical system may dynamically compute and display pressure information relating to the deviations and/or fluctuations of the IOP during the procedure. The pressure information may include a pressure metric or index that represents a total amount of deviation from the set point IOP during the procedure. The surgical system may provide the pressure metric to a surgeon or a user of the surgical system. The pressure metric may also be utilized by the surgical system to minimize the number of deviations of the IOP during the procedure such that the pressure metric is as close to zero as possible. The pressure metric or index reached at the end of the surgical procedure may be stored for post-operative assessment of the success of the surgical procedure.

Further, the surgical system monitors IOP levels during a surgical procedure and may also provide feedback to the user when a threshold value is reached during the procedure. The surgical system may monitor the IOP during a procedure and provide a notification if monitored IOP levels are outside of safe levels. For example, the surgical system may measure the IOP every 30-40 msec. A notification may be triggered if the monitored IOP is above a high pressure threshold continuously for a configured time period or is below a low pressure threshold continuously for a configured time period. In an example, the high pressure threshold may be a first threshold, and the low pressure threshold may be a second threshold, and the configured time period may be 3 seconds. Threshold values may be based on physiological IOP information, for example above 150 mmHg or below 14 mmHg for 3 or more seconds. The surgical system may enter a safe state, for example irrigation only, when a notification is triggered. For example, the pressure index may be compared to a threshold value and the surgical system may notify a user or surgeon when the threshold value has been reached or exceeded during the procedure. The threshold value may be defined by the surgical system or selected by the user. In some examples, the surgical system may notify a user or surgeon that the pressure index exceeded a threshold value after the procedure.

While the present surgical system may be used in various environments and applications, it will be discussed herein with a particular emphasis on an environment where a surgeon or health care practitioner performs. For example, the present surgical system may comprise an ocular surgical system that includes a graphical user interface (GUI), a surgical instrument or handpiece, and a surgical console or control module to control the surgical instrument.

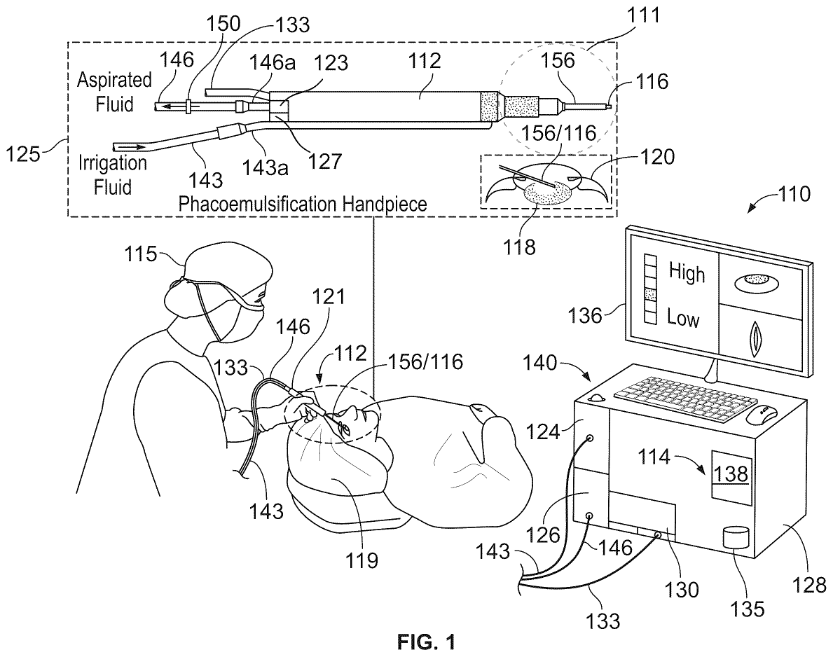

FIG. 1 is a schematic, pictorial view, along with an orthographic side view, of a phacoemulsification system 110, in accordance with an example of the present disclosure. As seen in the pictorial view of phacoemulsification system 110, and in the schematic side view inset 125, a phacoemulsification probe 112 (e.g., a handpiece) comprises a distal end 111 comprising a needle 116 and a coaxial irrigation sleeve 156 that at least partially surrounds needle 116 and creates a fluid pathway between the external wall of the needle and the internal wall of the irrigation sleeve, where needle 116 is hollow to provide an aspiration channel. Moreover, the irrigation sleeve 156 may have one or more side ports at or near the distal end to allow irrigation fluid to flow toward the distal end of the handpiece through the fluid pathway and out of the port(s).

Needle 116 is configured for insertion into a lens capsule 118 of an eye 120 of a patient 119 by a physician 115 to remove a cataract. While needle 116 (and irrigation sleeve 156) are shown in handpiece 125 as a straight object, any suitable needle may be used with phacoemulsification probe 112, for example, a curved or bent tip needle commercially available from Johnson & Johnson Surgical Vision, Inc., Irvine, CA, USA.

As shown in FIG. 1, during the phacoemulsification procedure, an irrigation pump 124, comprised in a console 128, pumps irrigation fluid from an irrigation reservoir (not shown) to irrigation sleeve 156 to irrigate the eye. The fluid is pumped via an irrigation tubing line 143 running from console 128 to an irrigation channel 143a of probe 112. In another example, pump 124 may be coupled with, or replaced by, a gravity-fed irrigation source such as a balanced salt solution bottle/bag. Fluid and waste matter (e.g., emulsified parts of the cataract) are aspirated via hollow needle 116 to a collection receptacle (not shown) by a processor-controlled aspiration pump 126, also comprised in console 128, using aspiration tubing line 146 running from aspiration channel 146a of probe 112 to console 128.

In the shown example, probe 112 may include an irrigation sensor 127 (e.g., pressure sensor) coupled with irrigation channel 143a and/or an aspiration sensor 123 (e.g., vacuum sensor) coupled with an aspiration channel 146a. Irrigation sensor 127 may be positioned anywhere along tubing line 143 or channel 143a. Likewise, aspiration sensor 123 may be positioned anywhere along tubing line 146 or channel 146a.

Channels 143a and 146a are coupled respectively with irrigation line 143 and aspiration line 146. Pumps 124 and 126 may be any pump known in the art (e.g., peristaltic pump, progressive cavity pump). Using sensors (e.g., as indicated by sensors 127 and/or 123), a processor 138 controls a flow rate of irrigation pump 124 and/or aspiration pump 126 to maintain IOP within prespecified limits.

The phacoemulsification system 110 includes an anti-vacuum surge (AVS) module 150, coupled with the probe 112, which prevents a sudden vacuum increase being transferred to the eye in case of an occlusion break. In some cases, typically to protect against a vacuum surge hazard, the system activates AVS module 150 (seen in inset 125) to disconnect aspiration channel 146a from line 146 and aspiration pump 126. AVS module 150 can be autonomous, with a processor inside AVS module 150 that receives and processes sensor readings and commands activation of module 150, or module 150 can be commanded from processor 138. To avoid vacuum surge inside the eye, the AVS module 150 very quickly (e.g., within a few tens of milliseconds) cuts off the aspiration line 146. However, blocking aspiration indefinitely by closing the valve of the AVS module 150 compromises “followability.” Followability is defined as the ability to attract cataract pieces to the phacoemulsification tip. Sufficient followability is important for efficient and safe phacoemulsification. An overly reactive system (or user) regarding vacuum surges as the tip is placed or moved within the capsular bag causes the system to fully interrupt suction (e.g., using the AVS mechanism) in cases of IOP drop, and such overreaction to AVS operation degrades followability.

To overcome or minimize the effects of a vacuum surge while maintaining acceptable followability, system 110 allows physician 115 to select a followability level. The processor activates AVS module 150 with a waveform predefined according to the selected followability.

It should be appreciated that the term anti-vacuum surge (AVS) module is used herein to refer to systems for preventing a sudden outflow of fluid from the eye chamber after an occlusion break due to vacuum build up in the aspiration line caused by an occlusion at the distal end of the needle, but such systems may be referred to equivalently by other terminology such as chamber stability/stabilization system (CSS).

As noted above, processor 138 may control the flow rate of irrigation pump 124 and/or aspiration pump 126, where one of the software modules running in processor 138 is a proportional-integral-derivative (PID) controller 114. Processor 138 estimates the IOP using readings from the irrigation pressure sensor 127 and an optional empirical offset (if the irrigation pressure is measured at the proximal end of handpiece or probe 112). Readings of sensor 123 give the vacuum level (also called sub-pressure) inside the aspiration channel.

Sensors 127 and 123 may be any sensor known in the art, including, but not limited to, a vacuum sensor or flow sensor. The sensor measurements (e.g., of pressure, vacuum, and/or flow) may optionally be taken close to the distal end of the handpiece where the irrigation outlet and the aspiration inlet are located, so as to provide processor 138 with an accurate indication of the actual measurements occurring within an eye, thus providing a short response time to a control loop comprised in processor 138. In an example, the same pressure sensor model is used to measure irrigation pressure and aspiration sub-pressure, using different sensor settings/calibrations.

As further shown, phacoemulsification probe 112 includes a piezoelectric crystal (not shown), coupled with a horn (not shown), that drives needle 116 to vibrate in a resonant vibration mode that is used to break a cataract into small pieces during a phacoemulsification procedure. Console 128 comprises a piezoelectric drive module 130, coupled with the piezoelectric crystal, using electrical wiring running in cable 133.

Processor 138 may receive user-based commands via a user interface 140, which may include setting a vibration mode and/or frequency of the piezoelectric crystal, and setting or adjusting an irrigation and/or aspiration rate of the irrigation pump 124 and aspiration pump 126. Optionally, user interface 140 includes a foot pedal. Processor 138 may receive user based commands via a user interface 140, which may include stroke amplitude settings for the needle 116 and commands for initiation of irrigation and/or aspiration. In an example, the physician uses a foot pedal (not shown) as a means of control. For example, a foot pedal may have a treadle that is moveable in a pitch direction and the available pitch travel may be divided into multiple functionality zones or positions. Foot pedal position 1 (FP1) activates only irrigation, foot pedal position 2 (FP2) activates both irrigation and aspiration, and foot pedal position 3 (FP3) adds needle 116 vibration. Additionally, or alternatively, processor 138 may receive user-based commands from controls located in a handle 121 of probe 112.

Some or all of the functions of processor 138 may be combined in a single physical component or, alternatively, implemented using multiple physical components. These physical components may comprise hard-wired or programmable devices, or a combination of the two. In some examples, at least some of the functions of processor 138 may be carried out by suitable software stored in a memory 135 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

In order to determine the IOP within a patient's eye during surgery, the console 128 of the phacoemulsification system 110 may be configured to determine and/or receive a variety of variables, parameters, and/or conditions about the eye treatment system 10. For example, the console 128 may determine a setpoint IOP based on input from a surgeon or user. The set point IOP may be a clinical setting rather than a physiological setting or threshold which can vary from patient to patient. In some examples, the setpoint IOP may be retrieved by the console 128 from memory.

The console 128 may utilize the inputs and parameters in an algorithm to determine the IOP before the surgery begins and/or to provide the IOP during and/or after surgery. The algorithm may be performed by the console 128 and may take into account one or more of the following parameters, such as bottle height, tip size, sleeve size, aspiration rate, vacuum rate, length and compliance metrics of various tubing used in the system, pump rate, etc. Other parameters for consideration in the algorithm are envisioned within the scope of this disclosure. For example, the IOP may be a function of static IOP and/or dynamic IOP measurements. As such, the IOP may be segmented into static and dynamic IOP during a phacoemulsification procedure, the static IOP being primarily impacted by the fluid inflow with small amount of outflow and the dynamic IOP being primarily impacted by fluid outflow.

The console 128 may monitor the IOP of the eye to maintain the stability of the IOP of the eye during a surgical procedure. For example, the console 128 may evaluate the IOP of the eye and calculate pressure information, such as the IOP. Since deviations and/or fluctuations of the IOP of the eye may cause complications, such as structural damage (e.g., posterior capsule rupture, ocular hypertension, and glaucoma), the console may be configured to actively monitor, evaluate, and/or control the IOP of the eye. In active control, the pressure in the eye is estimated and/or monitored with one or more sensors and the irrigation may be controlled by estimating the error between the set point IOP and real-time IOP measurements. In some examples, the console 128 may passively control the IOP of the eye. In passive control, the IOP in the eye may be controlled by the height of the solution bottle, and thus, the IOP may be controlled without the need of an external power supply.

The console 128 may be configured to monitor and evaluate the deviations, variations, and/or fluctuations of the IOP within the eye of the patient. The console 128 may generate metrics or information relating to the IOP within the eye based on the evaluation of the deviations, variations, and/or fluctuations of the IOP. For example, the console 128 may dynamically compute pressure information relating to the deviations and/or fluctuations of the IOP during the surgical procedure.

The pressure information may include a pressure metric or index that represents a total amount deviation from a set point IOP during the surgical procedure. The surgical system may provide or display the pressure metric to a user of the surgical system. For example, the pressure metric may provide an aggregated view of the overall chamber stability to assist a surgeon with IOP management (occluded and un-occluded) and CSS (post-occlusion surge) events or features. The pressure metric may also be utilized by the console 128 to control or minimize the amount of deviations and/or fluctuations of the IOP of the eye during the procedure, such that the pressure metric is as close to zero as possible. The pressure metric reached at the end of the procedure may be stored for post-operative assessment of the success of the surgical procedure.

The console 128 may calculate the pressure metric or index based on the following equation: Eq. 1

$$Pressure ( Index ) = {\sum }_{{t}_{6} ( surgery start )}^{t ( surgery end )} ABS ( P ( measured ) - P ( setpoint ) ) / Δ t for foot pedal position >= 1 and <= 3$$

wherein the foot pedal position >=1 and <=3, t₀ is the time at the surgery start, and t is the time at the surgery end.

The console 128 may be configured to calculate the pressure metric or index with respect to the set point IOP for phaco, irrigation/aspiration (I/A), and vitrectomy surgical modes. The pressure metric may track deviations and/or changes of the IOP of the eye within a particular surgical mode as well as the overall stability of the patient's eye across the surgical modes.

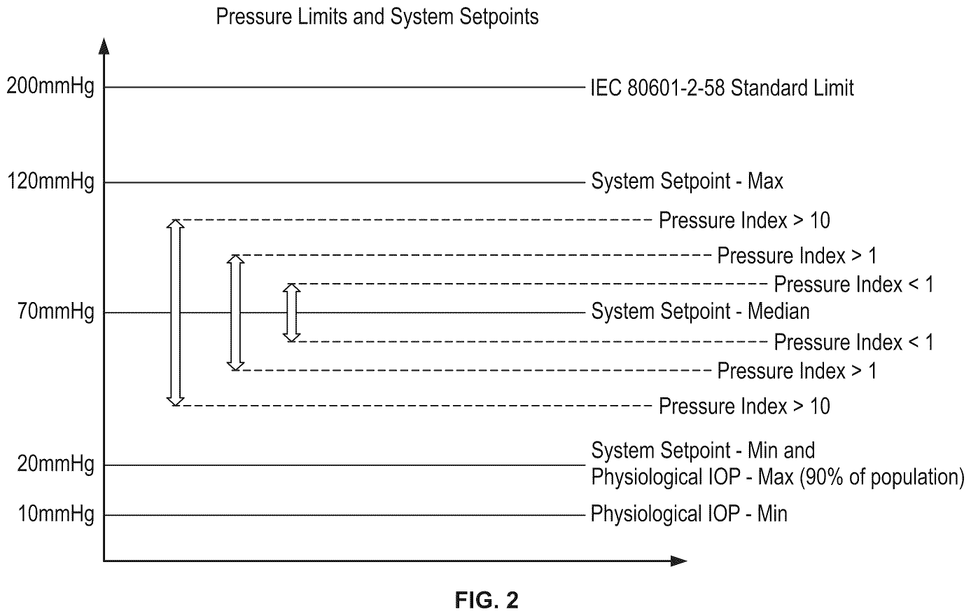

The pressure metric or index may be calculated each time the foot pedal is in position 1 or higher in the phaco, I/A, and vitrectomy surgical modes. The value of the pressure metric may increase as deviations and/or fluctuations from the set point IOP increases. FIG. 2 illustrates an example of the pressure limits and setpoints for an example system. As shown in FIG. 2, the set point IOP may be 70 mmHg with a working range of the IOP between 20 mmHg and 120 mmHg during a surgical procedure.

When the console 128 determines minimal deviations from the set point IOP during a surgical period, the pressure metric or index may be low, medium, or high. In the following examples of a low, medium, and high pressure metric or index, the metric or index is calculated over time as shown in Eq. 1. Thus, a deviation over a setpoint, even a high deviation, that is a short burst may not result in a change in the metric or index while the same deviation over the setpoint that is sustained for a longer period of time during a surgical procedure may result in a change in the metric or index. For example, a low pressure metric or index may have a value in a range of 0 to 1. It should be understood that different surgical modes may have different threshold values due to a difference in the tip/sleeve used for the respective surgical mode. A pressure index may be calculated with respect to an IOP setpoint for PHACO, I/A, and Vitrectomy surgical modes. This allows the index to track pressure changes within the given surgical more in addition to overall stability across the surgical modes. The pressure index may be calculated each time the foot pedal is in position 2 (FP2) or higher in the respective surgical mode. In an example, measurements may indicate a high pressure in the eye, for example pressure greater than 20 mmHg (threshold) for PHACO and IA. In another example, IA may have a threshold value of 23 mmHg. In an example embodiment, the threshold may be calculated as the current pressure plus/minus a set value when activating the irrigation and aspiration, for example entering FP2. Measurement may be stopped when the foot pedal is released or returned to FP1 and the handpiece is not in the eye.

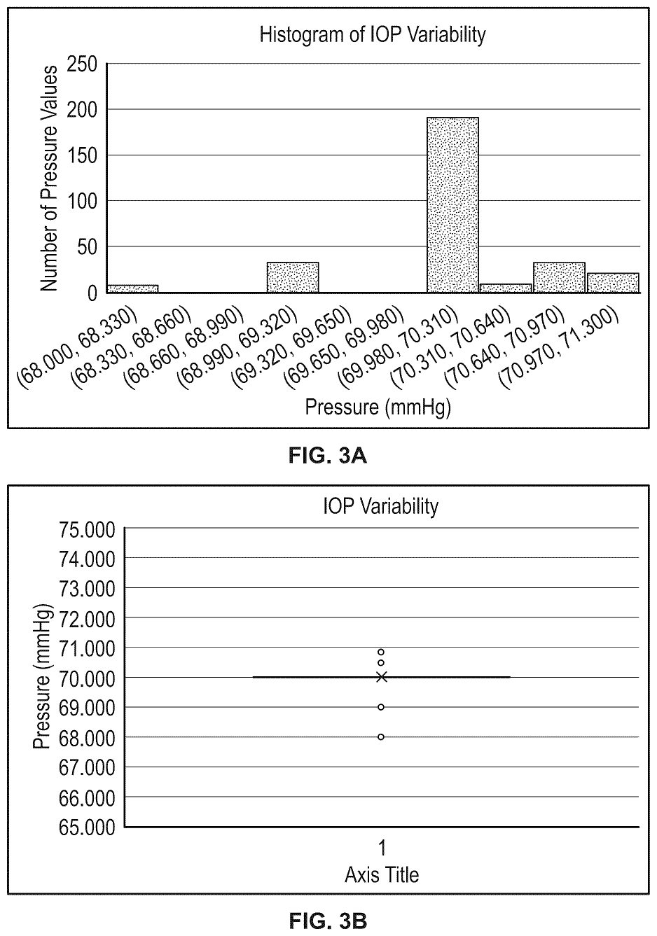

It is noted that parameters other than foot pedal position may be used to trigger the pressure index algorithm to begin calculating a pressure difference from the setpoint. For example, in addition to foot pedal position, one or more parameters detecting whether the handpiece is in the eye or outside in the air may be used to trigger the pressure index algorithm. For example, detecting that the handpiece is in the eye and entering FP2, may trigger the pressure index algorithm to start calculating a pressure difference from the setpoint. However, entering FP 2 while detecting the handpiece is not in the eye will not trigger the pressure index algorithm to start the calculation. In one example, the pressure metric or index may be based on IOP measurements performed during a surgical procedure as shown in the histogram of FIG. 3A and the variability of the IOP measurements from a set point (e.g., 70 mmHg) as shown in the graph of FIG. 3B.

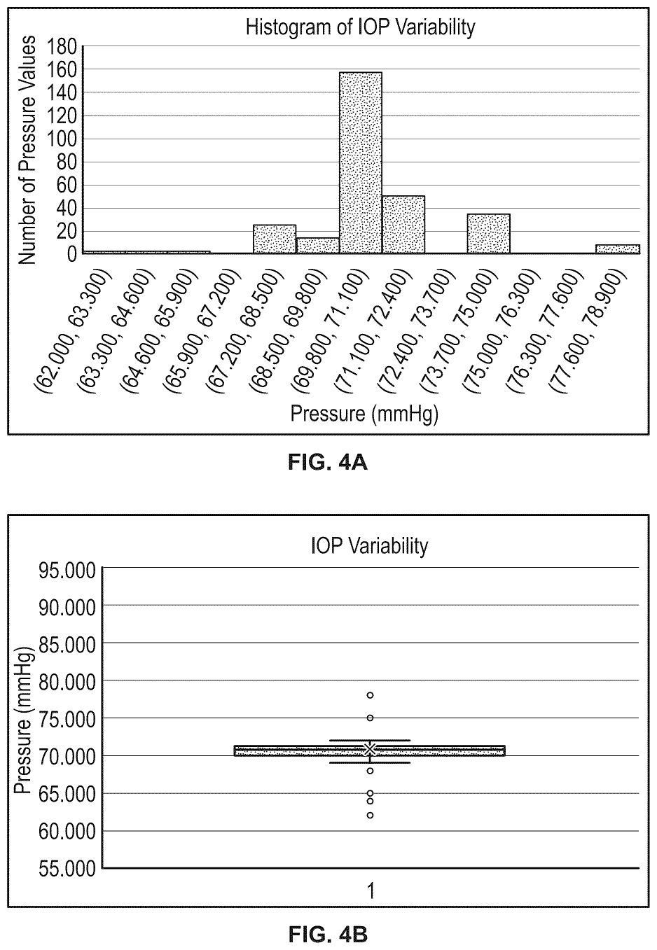

In another example, when the console 128 determines deviations from the set point IOP (e.g., +/−20 mmHg) during a surgical procedure, the pressure metric or index may have a medium value (e.g., median pressure index). FIG. 4A shows a histogram of IOP measurements performed during a surgical procedure. The variability of the IOP measurements from a set point (e.g., 70 mmHg) are shown in the graph of FIG. 4B.

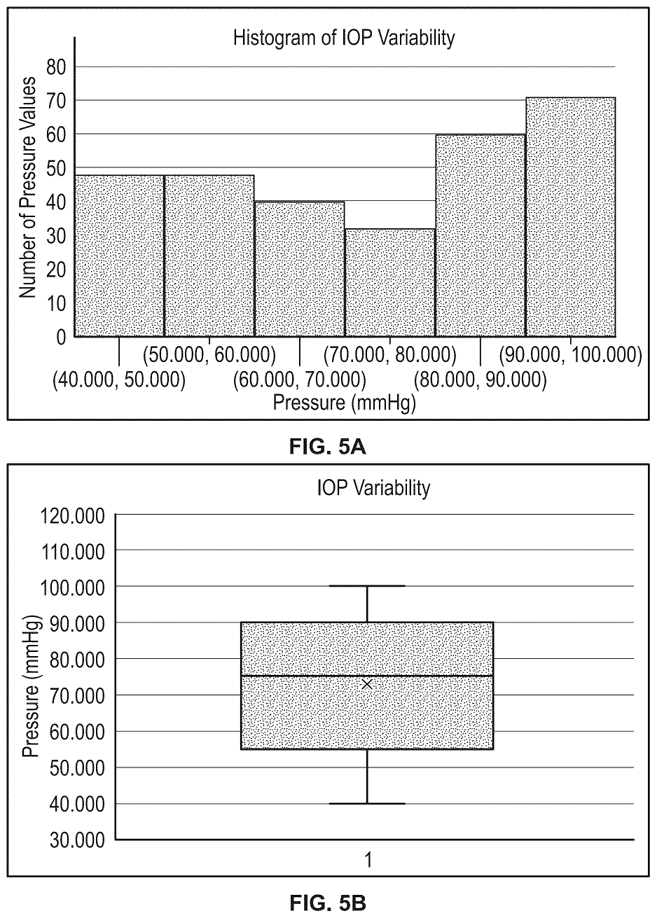

In another example, when the console 128 determines deviations from the setpoint IOP+/−30 mmHg during a surgical procedure, the pressure metric or index may have a high value (e.g., high pressure index). As described above, a deviation from the setpoint, for example +/−30 mmHg, that is a short burst may not result in a higher value for the pressure metric or index, while the same +/−30 mmHg change over a continuously longer period of time during the surgical procedure will likely result in a higher value for the pressure metric or index. Thus, as shown in Eq. 1, the deviation(s) and the duration of the deviation(s) occurring during the surgical procedure are used to calculate the pressure metric or index. For example, a pressure metric or index with a high value may be greater than 10. The IOP measurements performed during a surgical procedure as shown in the histogram of FIG. 5A and the variability of the IOP measurements from a set point as shown in graph of FIG. 5B illustrate an example that may result in a pressure metric or index with a high value Further, the pressure metric or index will be zero when the foot pedal is in position 0 or the handpiece is outside of the eye. The pressure metric or index will also be zero when the surgical system is in a diathermy mode and when the surgical system implements one of the following functions: continuous irrigation, prime/tune, cup-fill, reflux, and venting.

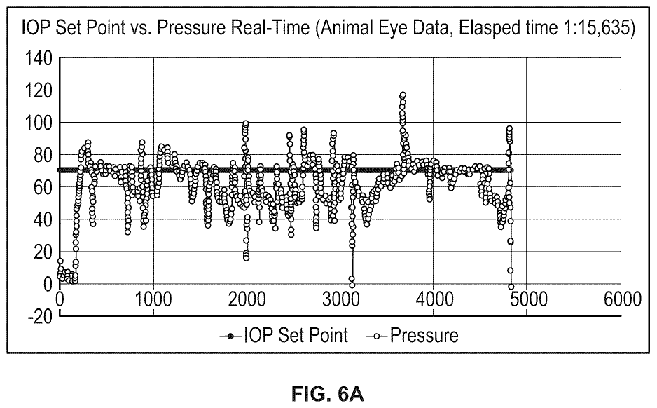

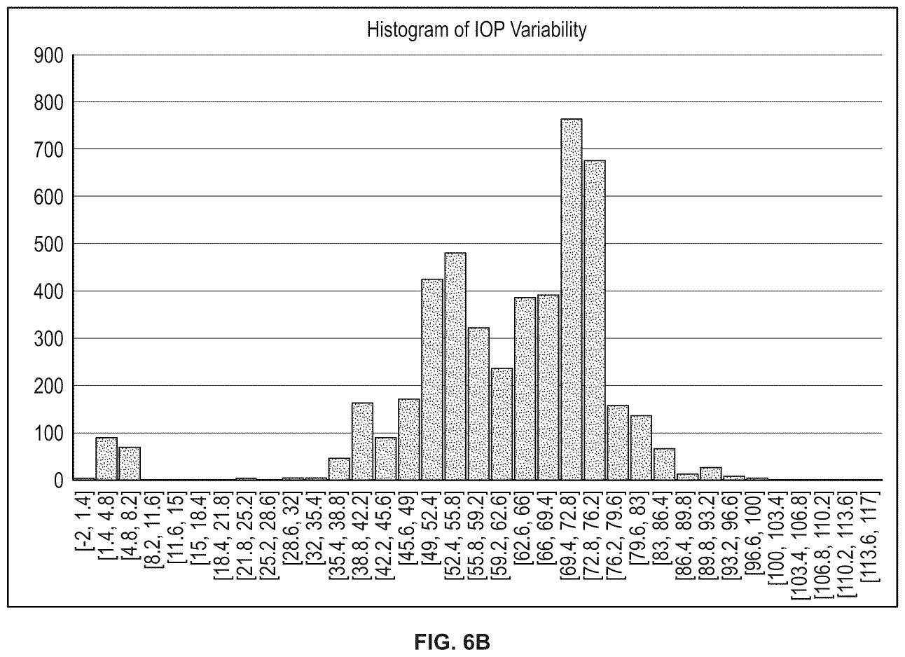

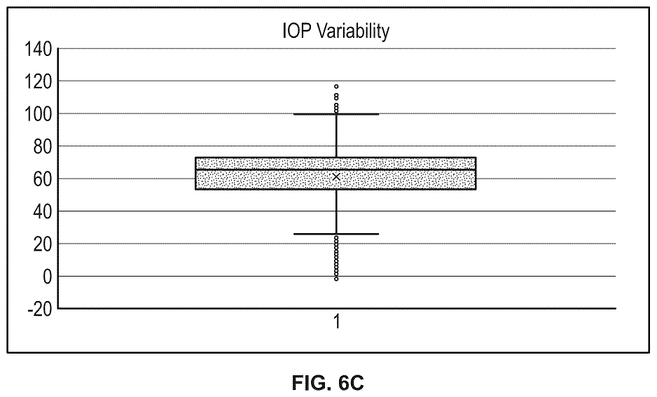

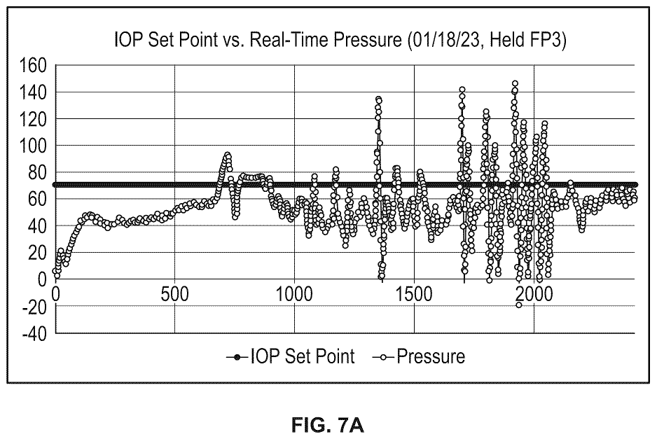

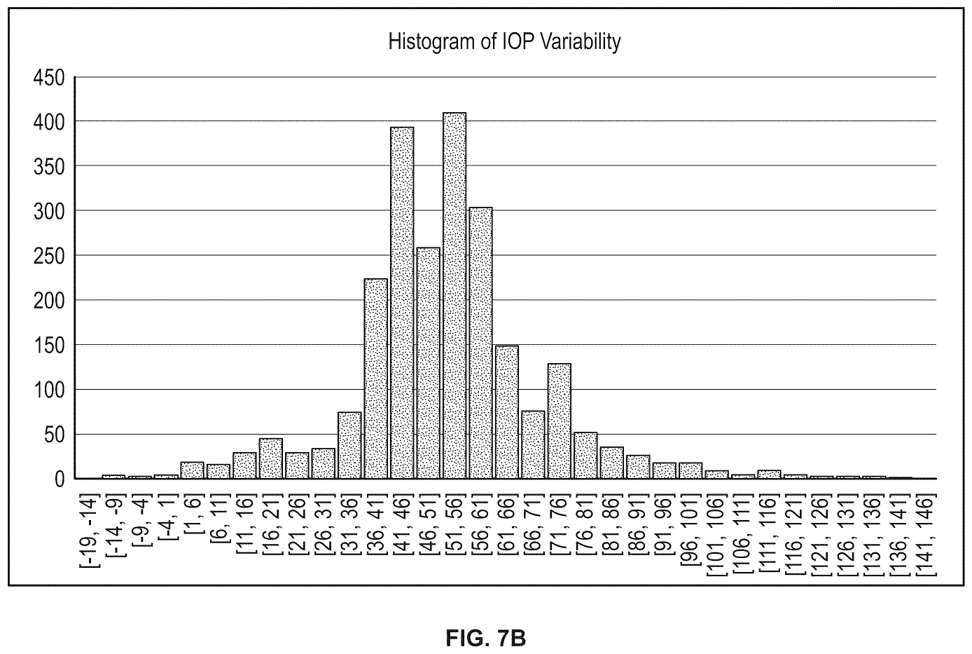

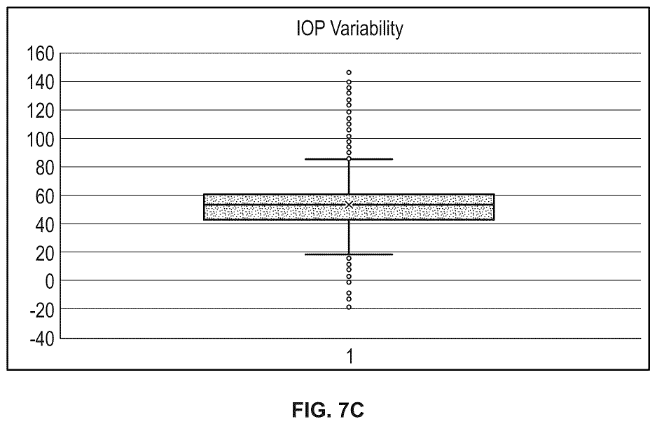

FIGS. 6A-6C illustrate the results of an experiment measuring the variability of IOP from a set point of 70 mmHg, foot pedal position (FP)>0, and the handpiece in the eye. In this experiment, the pressure index was determined to be equal to 0.7877. FIGS. 7A-7C illustrate the results of an experiment measuring the variability of IOP from a set point of 70 mmHg and FP=3. In this case, the pressure index was determined to be equal to 24.973032.

The console 128 may also provide feedback to the user when a threshold value is reached. For example, the pressure index may be compared to a threshold value and the console 128 may be configured to notify a user or surgeon, during and/or after the procedure, when the threshold value has been reached or exceeded. The threshold value may be defined by the console 128 or selected by the user.

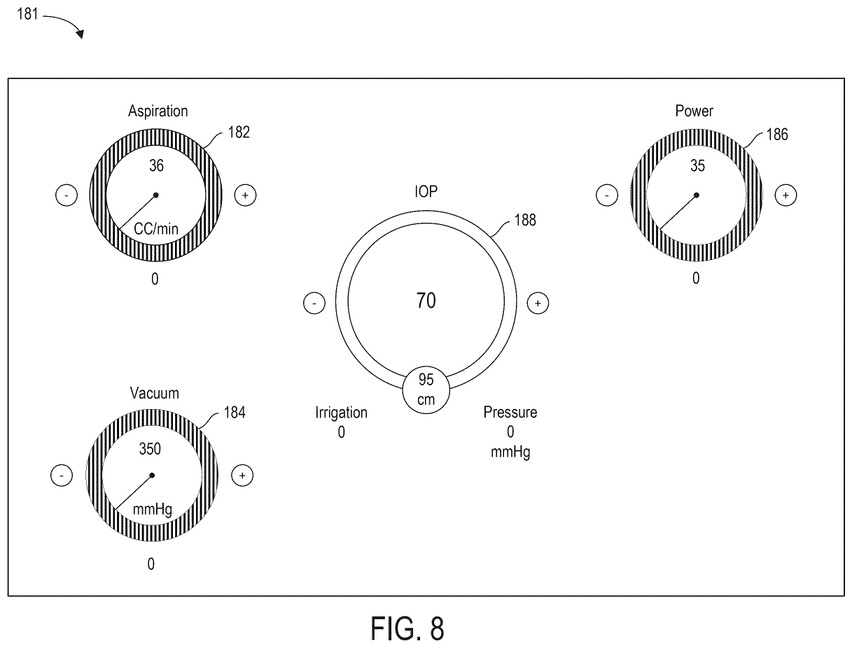

FIG. 8 shows an example graphical user interface (GUI) 181 displayed by the display device 136 (e.g., touchscreen, user interface, etc.) during a phacoemulsification procedure. The GUI may display various parameters, including aspiration levels 182, vacuum levels 184, power levels 186, and the IOP 188, The phacoemulsification parameters may be inputted or selected by the user via the GU L For example, the user may be presented with suggested prefilled parameters. Alternatively, the user may be presented with parameters previously saved by the user. In one example, the parameters may include, but are not limited to, a maximum power setpoint, a maximum aspiration setpoint, a maximum vacuum setpoint, a vacuum threshold, and/or power ramp time. The parameters inputted via the GUI and stored by the surgical console may be applied automatically when on automated phacoemulsification algorithm implemented by the console 128 (e.g., the controller of the console 128) is activated during the procedure or surgery.

The maximum power setpoint may be the maximum power percentage (from 0-100%) output through the handpiece 125 of the system. The maximum aspiration setpoint may be a maximum aspiration flow, from 0-80 cc/minute output through the handpiece 125. The max vacuum setpoint may be a maximum, from 0-700 mmHg, pull in the aspiration line to pull and remove lens particles from the eye. For example, the vacuum threshold may be the threshold that dictates the vacuum level that signifies a “full occlusion.” which signals the system to apply more power, for example starting from 35% of the maximum power setpoint to the maximum power setpoint. Power ramp time may specify how fast the power will ramp up (e.g., from 35% of the max power setpoint to the max power setpoint). The ramp time may have several operational settings, for example 0.5 seconds, 1 second, and 2 seconds.

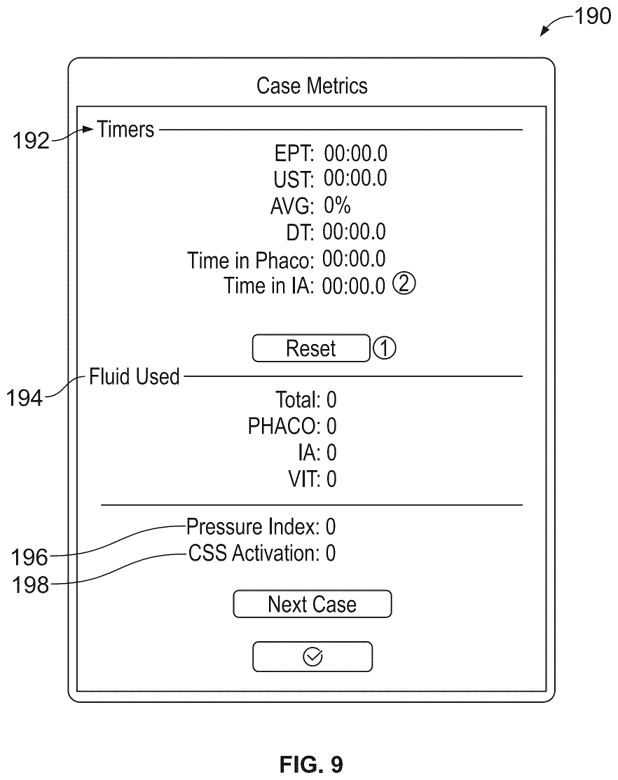

FIG. 9 shows an example graphical user interface (GUI) 190 displayed by the display device 136 (e.g., touchscreen, user interface, etc.) during a phacoemulsification procedure. The GUI may display various parameters, including timing information 192 (e.g., effective phaco time (EPT), docking time (DT), ultrasound time (UST), time in phaco mode, and time in I/A mode), fluid information 194 (e.g., an amount of fluid used in each surgical mode), a pressure index 196, and CSS activation 198.

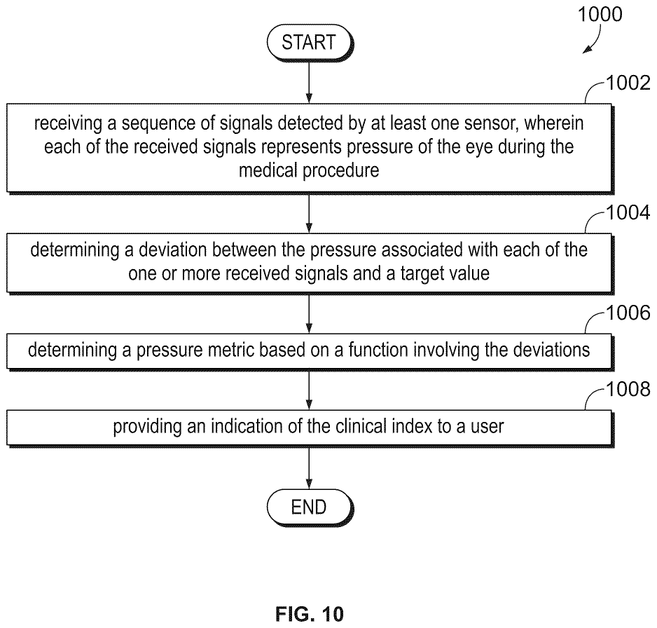

FIG. 10 is a flow chart of a method 1000 for monitoring pressure information during a medical or surgical procedure performed on an eye of a patient, according to an example implementation. The method 1000 represents an example method that may include one or more operations as depicted by one or more blocks 1002-1008, each of which may be carried out by the system shown in FIG. 1, among other possible systems. In an example implementation, a surgical system (e.g., phacoemulsification system 110) performs the illustrated operations, although in other implementations, one or more other systems can perform some or all of the operations.

Those skilled in the art will understand that the flow charts described herein illustrates functionality and operations of certain implementations of the present disclosure. In this regard, each block of the flowcharts may represent a module, a segment, or a portion of program code, which includes one or more instructions executable by one or more processors for implementing specific logical functions or steps in the processes. The program code may be stored on any type of computer readable medium, for example, such as a storage device including flash memory, EEPROM, a disk or hard drive.

In addition, each block may represent circuitry that is wired to perform the specific logical functions in the processes. Alternative implementations are included within the scope of the example implementations of the present application in which functions may be executed out of order from that shown or discussed, including substantially concurrent or in reverse order, depending on the functionality involved, as would be understood by those reasonably skilled in the art. Within examples, any system may cause another system to perform one or more of the operations (or portions of the operations) described below.

At the outset of the surgical procedure, a user or surgeon can provide or input a setpoint or target IOP for an eye of a patient, via an interface (e.g., a graphical user interface), into a surgical system (e.g., phacoemulsification system 110 of FIG. 1). In some examples, the setpoint IOP can be pre-programmed or retrieved from a memory device. Once the surgical system is configured, the surgeon may insert a tip of a phacoemulsification probe (e.g., a needle with a sleeve) into the eye of the patient. The surgeon may operate the system to irrigate the eye with irrigation fluid and to aspire material from the eye. For example, a controller or processor of the system may cause fluid to be delivered to chamber of the eye via an irrigation line. While fluid flows through the irrigation line into the eye, the processor may cause fluid and material from the eye to be delivered into an aspiration chamber or reservoir via the needle and an aspiration line, so as to obtain constant pressure in chamber of the eye.

At block 1002, the method involves receiving a sequence of signals detected by at least one sensor of the system. For example, the processor may receive signals from one or more sensors (e.g., pressure sensors) indicative of the detected or measured pressure level (e.g., IOP) in the eye. Each of the received signals may represent the current IOP of the eye during the procedure.

At block 1004, the method involves determining a deviation between the pressure value (e.g., IOP) associated with each of the one or more received signals and a set point or target value (e.g., a set point or target IOP value). For example, the processor may receive output from the one or more sensors and compare the output to a setpoint or IOP. The setpoint IOP may be a target IOP that is desired to be maintained during the surgical procedure.

The processor may monitor and evaluate the deviations, variations, and/or fluctuations of the IOP within the patient's eye. For example, the processor may, collect and use the parameters and conditions relating to the operation of the surgical system to maintain the IOP within the patient's eye at the setpoint or target IOP. At block 1006, the method involves determining a clinical index (e.g., pressure metric or index) based on a function involving the deviations. For example, the processor may provide metrics or information relating to the IOP based on the evaluation of the IOP within the patient's eye. In some examples, the function further involves accumulating an absolute value of the deviations. In other examples, the function further involves integrating the deviations over the period of time.

The processor may dynamically compute pressure information relating to the deviations and/or fluctuations of the IOP during the procedure. The pressure information may include a clinical index (e.g., a pressure metric or index) that represents a total amount of deviations from the setpoint IOP during the procedure. The pressure metric or index may be utilized by the surgical system to minimize the amount of pressure deviations during the procedure such that the pressure metric index is as close to zero as possible.

At block 1008, the method involves providing an indication of the clinical index to a user. For example, the processor may provide the clinical index (e.g., a pressure index) to a surgeon or a user of the surgical system. The system may also display additional information about the system.

The examples described above are presented by way of example, and the present disclosure is not limited by what has been particularly shown and described hereinabove. Rather, the scope of the disclosure includes both combinations and sub-combinations of the various features described hereinabove, as well as variations and modifications thereof which would occur to persons skilled in the art upon reading the foregoing description. It should be understood that many variations are possible based on the disclosure herein. Although features and elements are described above in particular combinations, each feature or element can be used alone without other features and elements or in various combinations with or without other features and elements.

The following examples describe one or more aspects of one or more embodiments.

### Example 1

A surgical system (110) for monitoring pressure events of an eye (120) of a patient (119) during a phacoemulsification procedure. The surgical system (110) comprising: at least one sensor (127) configured to provide one or more signals corresponding to pressure of the eye (120) during the phacoemulsification procedure; and a controller (114) of a surgical console (128) configured to: receive the one or more signals from the at least one sensor (127); determine a deviation between a target value and the pressure associated with each of the one or more received signals; determine a pressure metric (196) based on a function involving the deviations over a period of time; and provide the pressure metric for (196) display on a graphical user interface (190).

### Example 2

The surgical system (110) of example 1, wherein determining the pressure metric (196) further includes accumulating an absolute value of each of the deviations over the period of time as shown in equation 1, for example.

### Example 3

The surgical system (110) of example 1 or 2, wherein determining of the pressure metric (196) further includes computing an integral of the deviations over the period of time.

### Example 4

The surgical system (110) of example 1 or 3, wherein the graphical user interface (190) is configured to receive the target value from a user, and wherein the target value comprises an intraocular pressure value (188) to be maintained in the eye (120) of the patient (119) during the phacoemulsification procedure.

### Example 5

The surgical system (110) of example 1 or 4, wherein the controller (114) is further configured to maintain the pressure (188) within the eye (120) at or near the target value during the phacoemulsification procedure.

### Example 6

The surgical system (110) of example 1 or 5, further comprising a surgical handpiece (125) coupled with at least one irrigation line (143) and at least one aspiration line (146).

### Example 7

The surgical system (110) of example 6, wherein the at least one sensor (123/127) is coupled with the at least one aspiration line (146) or the at least one irrigation line (143).

### Example 8

An ophthalmic surgical system (110), comprising: a phacoemulsification probe (112) for performing a phacoemulsification procedure, the phacoemulsification probe (112) having a needle and (116) sleeve (156) at its distal end (111), the needle (116) configured to be inserted into an eye (120) of a patient (119); a pressure sensor (127) configured to detect a pressure (188) in an eye (120) of a patient (119); and a processor (138) configured to: receive a first sequence of signals from the pressure sensor (127) over a period of time, wherein each of the received signals represents a pressure (188) within the eye (120) during the phacoemulsification procedure; determine a clinical index (196) based at least on a function of the received signals, the function involving a deviation of the pressure associated with each of the received signals from a threshold pressure; and provide to a user (115) of the surgical system (110) an indication of the clinical index (196).

### Example 9

The ophthalmic surgical system (110) of example 8, wherein each signal of the first sequence represents an intraocular pressure (188) within the eye (120) of the patient (196) at a point in time during the phacoemulsification procedure.

### Example 10

The ophthalmic surgical system (110) of example 8 or 9, wherein the function further involves accumulating an absolute value of the deviations over the period of time.

### Example 11

The ophthalmic surgical system (110) of example 8 or 10, wherein the function further involves computing an integral of the deviations over the period of time.

### Example 12

A method for monitoring pressure events during a medical procedure performed on an eye (120) of a patient (119), comprising: receiving a sequence of signals detected by at least one sensor (127), wherein each of the received signals represents pressure (188) of the eye (120) during the medical procedure; determining a deviation between the pressure associated with each of the one or more received signals and a target value; determining a clinical index (196) based on a function involving the deviations over a period of time; and providing an indication of the clinical index (196) to a user (115).

### Example 13

The method of example 12, wherein the function further involves accumulating an absolute value of the deviations.

### Example 14

The method of example 12 or 13, wherein the function further involves integrating the deviations over the period of time.

### Example 15

The method of example 12 or 14, wherein each signal of the sequence represents an intraocular pressure (188) within an eye (120) of the patient (119).

### Example 16

The method of example 12 or 15, wherein the target value comprises an intraocular pressure value (188) to be maintained in the eye (120) of the patient (119) during the medical procedure.

### Example 17

The method of example 12 or 16, further comprising displaying, on a graphical user interface (190), the clinical index (196).

What is claimed is:

1. A surgical system for monitoring pressure events of an eye of a patient during a phacoemulsification procedure, the surgical system comprising:
at least one sensor configured to provide one or more signals corresponding to pressure of the eye during the phacoemulsification procedure; and
a controller of a surgical console configured to:
receive the one or more signals from the at least one sensor;
determine a deviation between a target value and the pressure associated with each of the one or more received signals, wherein the deviation is associated with a duration of time;
determine a pressure metric based on a function involving one or more deviations determined over a period of time; and
provide the pressure metric for display on a graphical user interface.

2. The surgical system of claim 1, wherein determining the pressure metric further includes accumulating an absolute value of each of the one or more deviations over the period of time.

3. The surgical system of claim 1, wherein determining of the pressure metric further includes computing an integral of the one or more deviations over the period of time.

4. The surgical system of claim 1, wherein the graphical user interface is configured to receive the target value from a user, and wherein the target value comprises an intraocular pressure value to be maintained in the eye of the patient during the phacoemulsification procedure.

5. The surgical system of claim 1, wherein the controller is further configured to maintain the pressure within the eye at or near the target value during the phacoemulsification procedure.

6. The surgical system of claim 5, wherein the controller is further configured to:
monitor the pressure metric; and
provide a notification when the monitored pressure metric is above a first threshold value continuously for a configured time period or below a second threshold value continuously for the configured time period.

7. The surgical system of claim 1, further comprising a surgical handpiece coupled with at least one irrigation line and at least one aspiration line.

8. The surgical system of claim 7, wherein the at least one sensor is coupled with the at least one aspiration line or the at least one irrigation line.

9. An ophthalmic surgical system, comprising:
a phacoemulsification probe for performing a phacoemulsification procedure, the phacoemulsification probe having a needle and sleeve at its distal end, the needle configured to be inserted into an eye of a patient;
a pressure sensor configured to detect a pressure in an eye of a patient; and
a processor configured to:
receive a first sequence of signals from the pressure sensor over a period of time, wherein each of the first sequence of signals received represents a pressure within the eye during the phacoemulsification procedure;
determine a clinical index based at least on a function of the first sequence of signals received, the function involving a deviation of the pressure associated with each of the first sequence of signals received from a threshold pressure, wherein a respective deviation is associated with a duration of time; and
provide to a user of the ophthalmic surgical system an indication of the clinical index.

10. The ophthalmic surgical system of claim 9, wherein each signal of the first sequence represents an intraocular pressure within the eye of the patient at a point in time during the phacoemulsification procedure.

11. The ophthalmic surgical system of claim 9, wherein the function further involves accumulating an absolute value of one or more deviations over the period of time.

12. The ophthalmic surgical system of claim 9, wherein the function further involves computing an integral of one or more deviations over the period of time.

13. The ophthalmic surgical system of claim 9, wherein the processor is further configured to:
monitor the clinical index; and
provide a notification when the monitored clinical index is above a first threshold value continuously for a configured time period or below a second a threshold value continuously for a configured time period.

14. A method for monitoring pressure events during a medical procedure performed on an eye of a patient, comprising:
receiving one of more signals detected by at least one sensor, wherein each of the one or more signals received represents pressure of the eye during the medical procedure;
determining a deviation between the pressure associated with each of the one or more received signals and a target value, wherein the deviation is associated with a duration of time;
determining a clinical index based on a function involving one or more deviations determined over a period of time; and
providing an indication of the clinical index to a user.

15. The method of claim 14, wherein the function further involves accumulating an absolute value of the one or more deviations.

16. The method of claim 14, wherein the function further involves integrating the one or more deviations over the period of time.

17. The method of claim 14, wherein the target value comprises an intraocular pressure value to be maintained in the eye of the patient during the medical procedure.

18. The method of claim 17, further comprising:
monitoring the clinical index; and
providing a notification when the monitored clinical index is above a first threshold value continuously for a configured time period or below a second a threshold value continuously for a configured time period.

19. The method of claim 14, further comprising displaying, on a graphical user interface, the clinical index.
